(19) United States
(12) Plant Patent Application Publication
(10) Pub. No.: US 2002/0188978 P1
(43) Pub. Date: Dec. 12, 2002

(54) Chrysanthemum plant named 'MOZA PA 01'

(76) Inventor: Frank Moser, Alva, FL (US)

Correspondence Address: KNOBBE MARTENS OLSON & BEAR LLP, 2040 MAIN STREET, FOURTEENTH FLOOR, IRVINE, CA, 92614, US

(21) Appl. No.: 09/864,900

(22) Filed: May 24, 2001

(51) Int. Cl. A01H 5/00

(52) U.S. Cl. PLT/289

(57) ABSTRACT

A new and distinct Chrysanthemum plant variety characterized by a decorative type inflorescence, consistent flowering response to short days, blooming consistently after 49 days of short day length, vigorous growth habit, and consistent natural season flowering habit during late September.

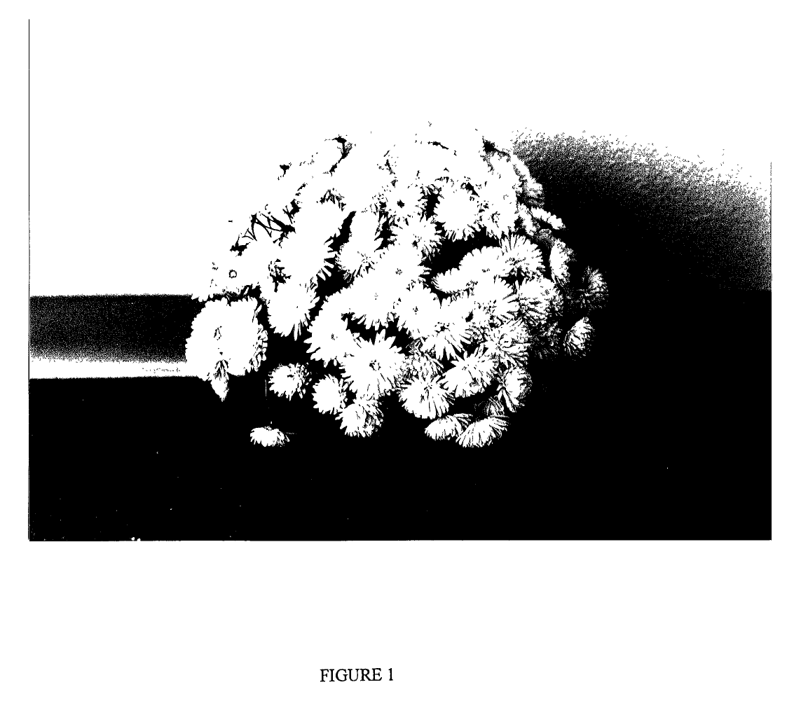

‘MOZA PA 01’: Table of R.H.S. Color Codes

| | | | |
|---|---|---|---|
| FOLIAGE | Leaf | young foliage upper surface | Near 137B |
| | | young foliage under surface | Near 137B |
| | | mature foliage upper surface | Near 137A |
| | | mature foliage under surface | Near 137A |
| | | venation upper surface | Near 138C |
| | | venation under surface | Near 138C |
| BLOOM | Ray Florets | upper surface at first opening | Near 6C |
| | | upper surface at maturity | Near 6A |
| | | upper surface at fading | Near 7B |
| | | under surface at first opening | Near 6C |
| | | under surface at maturity | Near 6A |
| | | under surface at fading | Near 7B |
| | | Pistillate: Stigma | Near 5A |
| | | Pistillate: Style | Near 5A |
| | Disc Florets | at first opening | Near 144B |
| | | at maturity | Near 144A |
| | | at fading | Near 143C |
| | | Staminate: Anther | Near 16A |
| | | Staminate: Pollen | Near 17B |
| | Peduncle | | Near 144A |
| | Inflorescence | | Near 147B |
| | Bud | | |
| | Sepals | upper surface | Near 137A |
| | | under surface | Near 137B |

### BACKGROUND OF THE INVENTION

The present invention relates to a new and distinct cultivar of Chrysanthemum plant, botanically known as Dendranthema morifolium and herein after referred to by the cultivar name ‘MOZA PA 01.’

This new cultivar originated from hybridization of a female parent Chrysanthemum cultivar (unpatented in the United States) with a male parent Chrysanthemum cultivar (unpatented in the United States). The new cultivar was discovered by the inventor in November 1998.

Asexual reproduction of the new cultivar by apical tip cutting and meristem tissue culture was performed in Oxnard, Calif. and has shown that the unique features of this new cultivar are stable and reproduced true to type on successive generations.

### SUMMARY OF THE INVENTION

The cultivar ‘MOZA PA 01’ has not been observed under all possible environmental conditions. The phenotype may vary somewhat with variations in environment such as temperature, daylength, and light intensity, without, however, any variance in genotype.

The following traits have been repeatedly observed and are determined to be the unique characteristics of ‘MOZA PA 01.’ These characteristics in combination distinguish ‘MOZA PA 01’ as a new and distinct Chrysanthemum: 1. Decorative type inflorescence: 2. Consistent flowering response to short days, blooming consistently after 49 days of short day length; 3. Vigorous growth habit; and 4. Consistent natural season flowering habit during late September.

Plants of the new cultivar are similar to plants of the female parent cultivar in most horticultural characteristics, however plants of the new cultivar differed from plants of the female parent cultivar in the following characteristics: 1. The new cultivar has different color ray florets; 2. The new cultivar has a larger diameter bloom; 3. The new cultivar is shorter; and 4. The new cultivar has a more flexible plant shape.

Plants of the new cultivar are similar to plants of the male parent cultivar in most horticultural characteristics, however plants of the new cultivar differed from plants of the male parent cultivar in the following characteristics: 1. The new cultivar is taller; and 2. The new cultivar naturally blooms 2 weeks later.

Plants of the new cultivar are similar to plants of the commercial cultivar ‘CLEAGAR GF1’ (disclosed in U.S. Plant Patent No. 10,463) in most horticultural characteristics, however plants of the new cultivar differed from plants of the commercial cultivai in the following characteristics: 1. The new cultivar has larger diameter blooms; 2. The new cultivar has more blooms per flowering branch; 3. The new cultivar is taller and wider; and 4. The new cultivar has a more flexible plant habit.

### BRIEF DESCRIPTION OF THE PHOTOGRAPHS

The photograph shows a plant of the new cultivar grown in a 1-gallon container. A single cutting was propagated in the container.

### DETAILED BOTANICAL DESCRIPTION

In the following description, color references are made to the Royal Horticultural Society Colour Chart except where general terms of ordinary dictionary significance are used. The following observations and measurements describe plants grown in a commercial filed on Oxnard, Calif. in the month of September 2000. The growing conditions range from 60 ° to 65 ° F. at night to 70° to 85° during the day. Measurements and numerical values represent averages of typical flowering types. Botanical classification: Dendranthema morifolium cultivar ‘MOZA PA 01’Commercial classification: Garden-type Chrysanthemum Parentage: Hybridization of female parent Chrysanthemum cultivar (unpatented in the United States) with a male parent Chrysanthemum cultivar (unpatented in the United States)

PROPAGATION

Time to Rooting.—7 to 14 days at approximately 21° C.

Rooting habit.—Fine, fibrous

PLANT

Growth Habit.—Mounding herbaceous perennial

Height.—Approximately 25 cm

Growth rate.—Moderate

Branching Characteristics.—Free Branching

Length of Lateral Branches.—Approximately 20 cm

Number of leaves per lateral branch.—Approximately 21

FOLIAGE

Leaf.—Arrangement: Alternate Average Length: Approximately 6.4 cm Average Width: Approximately 4.0 cm Shape of blade: Ovate Apex: Acuminate Base: Attenuate Attachment: Stalked Margin: Serrate Texture of top surface, Lightly pubescent Texture of bottom surface: Lightly pubescent Color: youngfoliage upper side: Near 137B young foliage under side: Near 137B mature foliage upper side: Near 137A mature foliage under side: Near 137A venation upper side: Near 138C venation under side: Near 138C

BLOOM

Inflorescence.—Flowering habit: Induced by darkness period greater than 13.5 hours, approximately 49 days of appropriate day length required to induce and develop blooms Inflorescence form: Decorative Natural flowering season: Approximately mid-September Number of inflorescences per stem: Approximately 16 Inflorescence diameter: Approximately 4.5 cm Inflorescence depth: Approximately 1.2 cm Inflorescence longevity on plant: Approximately 21-29 days Persistence: Persistent Ray Florets: Texture: Shiny Average Number perflower: Approximately 140 Shape: Oblanceolate Margin: Entire Apex: Obtuse Length: Approximately 2.0 cm Width: Approximately 4 cm Color:

upper surface at first opening: Near 6C upper surface at maturity: Near 6A upper surface at fading: Near 7B under surface at first opening: Near 6C under surface at maturity: Near 6A under surface at fading: Near 7B

Disc florets.—Appearance: Shiny Texture: Smooth Average number per flower: Approximately 30 Shape: Cylindric Apex: Obtuse Average Length: Approximately 0.5 cm Average Width: Approximately 0.3 cm Color: at first opening: Near 144B at maturity: Near 144A at fading: Near 143C

Peduncle.—Length: at terminal end (shortest): Approximately 3 cm at lateral end (longest): Approximately 6.54 cm Strength: Moderate Color: Near 144A Habit: Erect

Inflorescence bud.—Length: Approximately 0.3 cm Diameter: Approximately 0.4 cm Form: Globular Color: Near 147B

Sepals.—Appearance: Matte Texture: Lightly pubescent Number: Approximately 24 Shape: Ovate Margin: Entire Apex: Acute Color: upper side: Near 137A under side: Near 137B

REPRODUCTIVE ORGANS

Ray Florets.—Pistillate: Number ofpistils perflower: 1 Stigma shape: 2 branched Stigma color: Near 5A Style color: Near 5A Style length: Approximately 0.3 cm

Disc Florets.—Pistillate: Number ofpistils per flower: 1 Stigma shape: Cylindric Style length: Approximately 0.3 cm Staminate: Number ofstamens per flower: Approximately 5 Anther shape: Tubular Anther color: Near 16A Pollen color: Near 17B

OTHER CHARACTERISTICS

Disease Resistance.—Neither resistance nor susceptibility observed

What is claimed is:

1. A new and distinct variety of Chrysanthemum plant named ‘MOZA PA 01’ as herein illustrated and described.
